(19) United States
(12) Patent Application Publication
(10) Pub. No.: US 2005/0121035 A1
(43) Pub. Date: Jun. 9, 2005

(54) Respiratory apparatus for compressed-air breathing equipment

(76) Inventor: Jan Martin, Berlin (DE)

Correspondence Address: James G Uber;Mine Safety Appliances Company, PO Box 426, Pittsburgh, PA, 15230-0426, US

(21) Appl. No.: 10/492,769

(22) PCT Filed: Sep. 10, 2002
(86) PCT No.: PCT/DE02/03421

(30) Foreign Application Priority Data
Oct. 19, 2001 (DE) 101 52 454.4

(51) Int. Cl. A62B 7/00; A61M 16/00

(52) U.S. Cl. 128/205.24; 128/204.18

(57) ABSTRACT

A oxygen system includes a casing (1) that houses a valve seat (4) with an air inlet duct (6) arranged in axial direction, a shutting part (7) associated with the air inlet duct at the valve outlet, and a driving motor (8). A pressure sensor (15) for measuring the pressure in the respective phase of breathing is located in a measuring chamber (2) connecting the air inlet duct and the air outlet duct (3) that is formed in the casing and can be connected to a respirator mask. The pressure conditions prevailing during inhalation and exhalation control the drive motor that is connected by a controller to the shutting part of the valve unit, and regulate the air supply to the respirator mask. In addition to the air control that exactly matches respiration, the device is small and compact, requires few mechanical components, and is resistant to impact stress.

The invention relates to an oxygen system for compressed air breathing apparatuses with a respiration-controlled valve unit for controlling the air supply to a respirator mask.

Oxygen systems of this type have been known for a long time. They are installed between the pressure reducing valve of a compressed air reservoir and a user's breathing mask and ensure provision of a specific respiratory air quantity at a pressure suitable for the human system. In known oxygen systems, the valve unit is operated to release air and to control air supply from the pressure reducer to the respirator mask by means of a control membrane that is moved due to the negative pressure produced by the user when inhaling.

As membrane control is purely mechanical, multiple mechanical components are required to interact. This requires a large space while control of the air quantity supplied to the user is imprecise and unsteady and cannot be adjusted to the user's special needs. In addition, an oxygen system designed with a mechanical membrane control is highly sensitive to external mechanical influences that can result in irregular air supply or even interruptions of air supply to the user.

It is therefore the problem of the invention to design an oxygen system of the type mentioned above that is compact in size and ensures trouble free functioning and control of air supply that adjusts to the specific respiratory needs of the user.

This problem is solved according to the invention by the oxygen system comprising the characteristics described in claim 1.

The dependent claims disclose further characteristics and advantageous improvements of the invention.

The invention is based on the concept that a driving motor controlled by a pressure sensor operates the valve unit or releases the air supply to the user during inhalation and interrupts the air supply during exhalation. A shutting part associated with the outlet opening in the valve seat opens or closes the outlet opening more or less depending on the pressure in the oxygen system during inhalation and exhalation that a pressure gauge transmits to the driving motor via a controller.

The oxygen system according to the invention includes a casing that houses a valve seat with an air inlet duct in central position, a shutting part assigned to the air inlet duct, and a driving motor for the shutting part. The pressure sensor that measures the pressure in the respective breathing phase based on which the controller controls the driving motor according to a predefined control characteristic is located in a measuring chamber that connects the air inlet duct and the air outlet duct formed inside the casing and connected to the respirator mask.

Control of the air supply is very steady and adjusted to the user's needs or the breathing conditions. Only few mechanical components are required due to valve operation using a driving motor controlled by measured pressure. This increases service life and reduces susceptibility to failure. The unit size is considerably smaller as compared to known devices. In particular, malfunctions due to mechanical impact on the oxygen system are generally eliminated. The noises known from mechanically controlled oxygen systems do not occur with the device according to the invention.

According to another important characteristic of the invention, the driving motor that is connected to the shutting part of the valve unit via a driving member rests on elastic supports. If unacceptably high air pressure acts on the shutting part, e.g. when the upstream pressure-reducing valve is defective, pressure can be compensated due to the elastic support of the motor and elastic mount of the shutting part.

Another advantageous embodiment of the invention features a manually adjustable valve seat with central air inlet that facilitates air supply by manual movement of the valve seat in the event of a malfunction of the shutting part motion.

Embodiments of the invention that lead to further advantageous improvements are explained in more detail with reference to the figures that show both the inhalation or open position of the valve and the exhalation or closed position of the valve. Wherein:

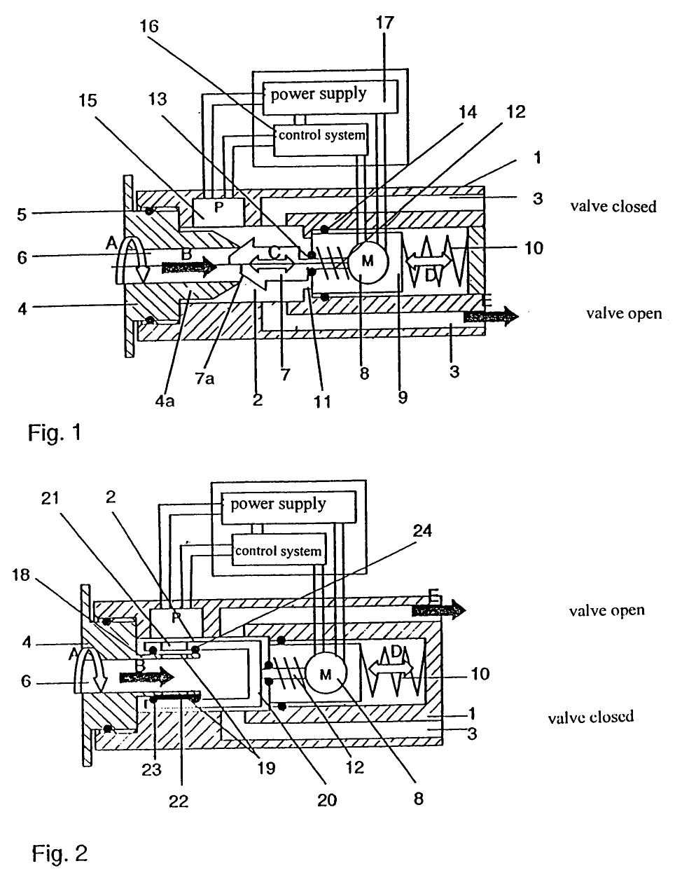

FIG. 1 shows a first embodiment of an oxygen system according to the invention with a translatorily moved shutting part for releasing and controlling the air supply to the user;

FIG. 2 shows a second embodiment of the oxygen system with a shutting member that controls air supply by a rotational movement;

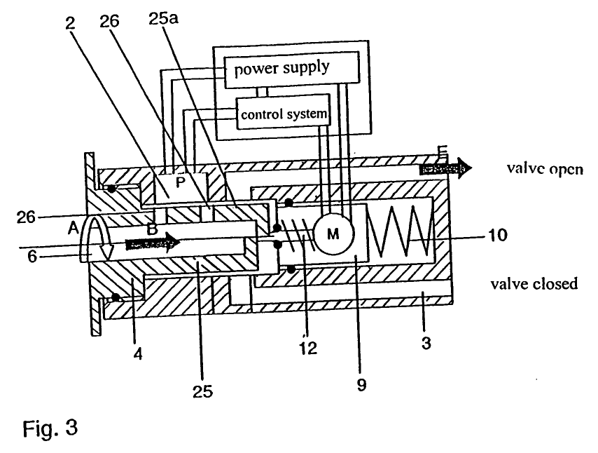

FIG. 3 shows a third embodiment of an oxygen system with an elastic shutting element that releases the air supply to the user via the respirator mask due to its change in length.

### 1^{st} EMBODIMENT

The embodiment of an oxygen system shown in FIG. 1 includes a casing 1 with a measuring chamber 2 that can be connected to the respirator mask via an air outlet duct 3 (not shown) on the air outlet side. A valve seat 4 that can be adjusted in axial direction as indicated by arrow A by manual rotational movement is located in the casing 1 on the air inlet side that is connected to a pressure reducer (not shown) via a medium pressure line. A packing ring 5 is provided for sealing purposes between the valve seat 4 and the casing 1. Air is supplied from the medium pressure line (not shown) along arrow B via an air inlet duct 6 running in axial direction inside the valve seat 4 that can be closed by a shutting part 7 that can be moved in axial direction (arrow C) using a driving motor 8. The shutting part 7 is designed as a shutting cone 7a to close off the opening section 4a on the front end of the valve seat 4. The driving motor 8 is housed in a drive casing 9 that is pressed towards the air inlet side (arrow D) against a stopper surface 11 through the action of a safety spring 10. The drive casing and a driving member (driving spindle) 12 are sealed towards the measuring chamber 2 by packing rings 13 and 14. A pressure sensor 15 is connected to the measuring chamber 2 that represents a connecting space between the air inlet duct 6 and the air outlet duct 3. The pressure sensor 15 is connected to the driving motor 8 by a controller 16. In addition, a power supply unit 17 is connected to the driving motor 8 and the pressure sensor 15.

The oxygen system described with reference to FIG. 1 operates as follows:

The figure shows the oxygen system in closed valve position in the upper half and in open valve position in the lower half. When the pressure sensor 15 detects a specific pressure drop in the measuring chamber 2 during the inhalation phase when the air inlet duct 6 is still closed (the shutting part 7 seals off the valve seat 4), the controller 16 sends a signal reflecting the quantity of pressure change to the driving motor 8 which retracts the shutting part (lower half of the figure). Air supplied via a medium pressure line (not shown) can thus flow through the air inlet duct 6, the measuring chamber (connecting space) 2, and the air outlet duct 3 (not shown). As the driving motor 8 is controlled based on the pressure conditions, air supply to the user can be adjusted exactly to the prevailing conditions.

The pressure sensor 15 controls the driving motor 8 in the exhalation phase, due to the rise in pressure, so that the shutting part 7 is moved towards the valve seat 4 and seals the valve seat 4 and no respiratory air can flow into the device.

The function of the safety spring 10 that acts on the drive casing 9 is to press the shutting part 7 and the entire drive case 9 back and to release the opening of the valve seat 4 for air to flow out when the oxygen system is under unacceptably high pressure, e.g. when the pressure reducer fails.

Another safety feature enables the user to manually unscrew the valve seat 4 from the casing 1. In this way the user can be supplied with respiratory air even if the drive motor 8 has failed and can no longer retract the shutting part 7 and open the air inlet duct 6.

2. 2^{nd} EMBODIMENT

The embodiment shown in FIG. 2 differs from the one described above in the design of the valve unit only. It consists here of a valve seat 4 with an inside cylinder 18 the wall of which comprises first through holes 19. The shutting part is designed as a closing pot 20 that encompasses the inside cylinder 18 and can be rotated around its longitudinal axis using the driving motor 8. The wall of the closing pot 20 comprises a second through hole 21 at the same level as the first through hole 19. Two packings 23 and 24 are provided on the perimeter of the inside cylinder 18 in such a way that the through holes are located between these.

In this embodiment, air is supplied to the respirator mask via the oxygen system as follows: the driving motor 8 rotates the closing pot 20 due to the pressure conditions in the measuring chamber 2 during inhalation transmitted from the pressure gauge 15 to the controller 16 and places its through hole 21 over the through hole 19 of the inside cylinder 18 (extension of the valve seat). The user is supplied with a quantity of air depending on the extent to which the second through hole 21 overlaps the first through hole 19 as a function of the measured pressure. FIG. 2 shows the open valve position in the upper half wherein the inhalation air flows along arrows B and E from the medium pressure line via the air inlet duct 6 the through holes 19 and 21, the measuring chamber 2, and the air outlet duct 3 to the respirator mask.

The lower half of FIG. 2 shows the completely closed valve position during exhalation wherein the driving motor 8 rotated the closing pot 20 due to increased pressure into a position in which the through holes 19 are sealed, shown as a sealing area in FIG. 2.

The action of the safety spring 10 is triggered by high pressure from the medium pressure line that moves the closing pot 20 in axial direction against the elastic force of the safety spring 10. This eliminates the sealing effect of the front packing 24, and air can flow out.

When the closing pot 20 cannot be rotated, e.g. due to a failure of the driving motor 8, and no respiratory air can flow to the user, this second embodiment includes the option of manually turning the valve seat 4 with the inside cylinder 18 so that one of the first through holes 19 as brought to an overlap with the second through hole 21 and respiratory air can flow to the respirator mask.

3. 3^{rd} EMBODIMENT

In the embodiment of the oxygen system shown in FIG. 3, the valve unit for releasing or blocking air supply is designed as a longitudinally adjustable valve element 25 with valve openings 26 integrated into its elastic section. Under the respective pressure conditions in the inhalation phase, the elastic valve element is stretched using the driving member 12 operated by the driving motor 8 so that the valve openings 26 are released and respiratory air can flow from the air inlet duct 6 via the valve openings 26, the measuring chamber 2, and the air outlet duct 3 to the user. A dosed quantity of air based on the pressure conditions measured by the pressure sensor 15 can be conducted to the mask in that the driving motor 8 stretches the elastic valve element 25 by a specific length and opens the valve openings 26.

As the motor rests on elastic support in this embodiment, too, air can flow out when the pressure at the air inlet duct is unacceptably high. Likewise, the valve seat 4 can be manually reset in the event of a drive failure to ensure air supply to the mask even when such a failure occurs.

1-12. (canceled)

13. In a regulator for a compressed air-breathing apparatus having a respiration-controlled valve unit housed in a casing for controlling a supply of air to a respirator mask, the improvement wherein the valve unit comprises: a valve seat; an air inlet duct; and a shutting part operated by a driving motor and wherein a pressure sensor is located in a measuring chamber and is electrically-connected to the driving motor via a controller for controlling the supply of air via a controller for controlling the supply of air based on a measured pressure in a measuring chamber; and wherein the measuring chamber connects the air inlet duct and an air outlet duct formed in the casing that is connected to the respirator mask.

14. The regulator according to claim 13, wherein the driving motor and a driving member linked with the shutting part is elastically held on a stopper surface in the casing against air pressure acting on the shutting part by an elastic support so that the shutting part can be adjusted automatically for releasing the air inlet duct when an unacceptably high pressure has built up.

15. The regulator according to claim 14, wherein the elastic support is a safety spring that presses the drive casing of the driving motor against the stopper surface.

16. The regulator according to claim 13, wherein the valve seat is manually adjustable and sealed in the casing so that it can be connected to the air outlet duct in the event of a failure that renders the shutting part inoperable.

17. The oxygen system according to claim 16 wherein the air inlet duct of the valve seat can be shut at a front outlet side opening by the shutting part being movable in longitudinal direction.

18. The regulator according to claim 17 wherein the valve seat can be adjusted in an axial direction by manual rotation.

19. The regulator according to claim 13 wherein the valve seat comprises an inside cylinder on its outlet side with through holes in the cylinder wall and a closing pot that functions as the shutting part and has a lateral through hole that laps over the inside cylinder, the closing pot being rotatable around its axis by the driving motor depending on the measured pressure conditions to allow air supply to the respirator mask when the through holes of the inside cylinder and the closing pot overlap partially or completely.

20. The regulator according to claim 19 wherein the closing pot, the driving member, and the driving motor can be moved in a longitudinal direction by the action of a high pressure, thereby disengaging a packing provided between the inside cylinder and the closing pot.

21. The regulator according to claim 19 wherein the inside cylinder can be rotated by manually rotating the valve seat to ensure a drive-independent supply of air to the respirator mask.

22. The regulator according to claim 13 wherein the shutting part comprises a pot-shaped elastic valve element connected to the valve seat that is stretched by the driving member and driven by the driving motor based on a pressure measured in the measuring chamber and thereby opens valve openings in the side walls to a greater or lesser extent, or closes them.

23. The regulator according to claim 22 wherein the elastic valve element can be elastically stretched by manually adjusting the valve seat in a longitudinal direction to ensure a drive-independent supply of air to the respirator mask.

24. The regulator according to claim 22 wherein the elastic valve element can be elastically stretched to open the valve opening when an unacceptably high pressure acts on the inlet.
